(19) United States
(12) Patent Application Publication
(10) Pub. No.: US 2006/0036287 A1
(43) Pub. Date: Feb. 16, 2006

(54) Defibrillator comprising a relay testing device

(76) Inventor: Heinz Bucher, Rothwell (DE)

Correspondence Address: PAULEY PETERSEN & ERICKSON, 2800 WEST HIGGINS ROAD, SUITE 365, HOFFMAN ESTATES, IL, 60195, US

(21) Appl. No.: 10/535,592

(22) PCT Filed: Nov. 17, 2003
(86) PCT No.: PCT/EP03/12855

§ 371 Date: May 19, 2005

(30) Foreign Application Priority Data
Nov. 19, 2002 (DE) 102 54 482.4

(51) Int. Cl. A61N 1/39

(52) U.S. Cl. 607/5

(57) ABSTRACT

A defibrillator including an output stage that has a high-voltage section and patient electrode connections which can be automatically connected by a coupling circuit to said high-voltage section via a relay, and that has a relay testing device. A reliable functioning of the defibrillator is ensured due to the provision of a discharging resistor device which can be automatically switched to instead of the patient electrode connections by the relay, and because the relay testing device is designed for testing the relay while involving the state of the adjacent discharging resistor device.

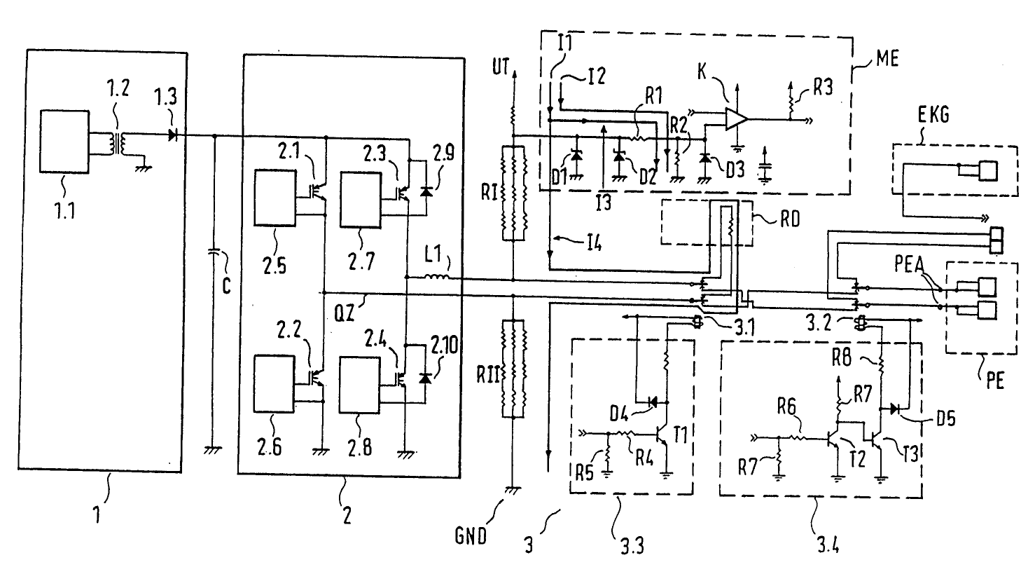

### BACKGROUND OF THE INVENTION

1. Field of the Invention

This invention relates to a defibrillator, particularly a portable external defibrillator, having an output stage, which has a high-voltage element and patient electrode connectors to which can be automatically connected with a coupling circuit via a relay, as well as a relay testing device.

2. Discussion of Related Art

A defibrillator is disclosed in European Patent Reference EP 0 946 956 B1. A method with a known defibrillator, by which a relay, arranged in a coupling circuit between a high-voltage element and patient electrodes, is tested and can be brought into an open position and into a closed position, in which the patient electrodes are connected with the high-voltage element. A test run is performed for testing the relay, in which a discharge of an energy storage element in the form of a capacitor, which stores the energy for the defibrillation pulse, is performed via the relay and the patient electrodes, and the voltage at the capacitor is measured. The voltage at the capacitor during the discharge is compared with a threshold voltage in order to determine the relay status. In various situations it is thus difficult to arrive at a dependable statement regarding the relay status.

An automatic external defibrillator (AED), in particular a portable one, with a specially embodied high-voltage element with an H-bridge and patient electrodes, which are connected in the transverse branch of the latter in series to form an inductive resistor (coil or equivalent component) is taught by German Patent Reference DE 100 65 104 A1. It is possible with this known defibrillator to generate bi-phased defibrillation pulses by an appropriate control of switching members of the H-bridge. However, there is no relay test mentioned.

### SUMMARY OF THE INVENTION

One object of this invention is to provide an external defibrillator, in particular a portable one, such as mentioned above but in which the testing of the coupling circuit can be performed as dependably as possible.

This object is achieved with a defibrillator having characteristics taught in this specification and in the claims. A discharge resistor arrangement exists, to which a switch can be automatically made by the relay, instead of to the patient electrode connectors, and the relay testing device is designed for testing the relay while incorporating the status of the connected discharge resistor arrangement.

Dependable conditions for testing the relay are created by the connected discharge resistor arrangement, so that a dependable evaluation of the relay status is achieved.

The testing of the coupling relay is dependable because the relay testing device has its own voltage supply for a test supply voltage, by which a current can be run through the relay for testing the relay with the connected discharge resistor arrangement, wherein a current from the high-voltage element is blocked. These steps contribute to unequivocal testing conditions, while a simple construction of the measuring device can be realized.

In a further embodiment of the relay testing device there is a measuring branch in which, with the discharge resistor arrangement connected or the discharge resistor arrangement disconnected, different voltages or measuring currents exist, which can be incorporated in the testing of the relay.

In this case measurement is more dependable if the measuring branch has a measurement amplification circuit for forming a measured value regarding a relay status.

Unequivocal statements are also assisted if the measurement amplification circuit has a comparator for comparing with a reference variable.

In one advantageous construction of the defibrillator with the relay testing device the high-voltage element has an H-bridge, which can be charged with high voltage by an energy storage device for a defibrillation pulse, and has controllable switching members in the H-legs, and the relay is arranged in series with an inductive resistor and on one side with the discharge resistor or on the other side with the patient electrodes connected to the patient electrode connectors in the transverse branch of the H-bridge.

In a further embodiment of the defibrillator with the relay testing device a further relay is integrated between the relay and the patient electrode connectors, by which the patient electrodes can be selectively connected with the high-voltage element or an EKG measuring device.

### BRIEF DESCRIPTION OF THE DRAWING

This invention is explained in greater detail in view of an exemplary embodiment and by making reference to the drawing, wherein the drawing shows a schematic diagram of an output stage of a defibrillator.

### DESCRIPTION OF PREFERRED EMBODIMENTS

The drawing figure shows an output stage of a defibrillator, in particular a portable external automatic defibrillator (AED), having a high-voltage element with a charging device 1 and an H-bridge 2, as well as a connected coupling circuit 3. The charging device has a charging element 1.1, for example an alternating voltage source with a transformer 1.2 connected therewith, for generating a high voltage, by which an energy storage element in the form of a storage capacitor C, or a storage capacitor arrangement, is charged for defibrillation with electrical energy via a charge diode 1.3 in a known manner. In its H-legs, respectively oriented toward the positive pole on the one side and toward the negative pole on the other side, the H-bridge 2 has respective switching members 2.1, 2.2, 2.3, 2.4, which are triggered by assigned trigger circuits 2.5, 2.6, 2.7, 2.8, such as described in greater detail in German Patent Reference DE 100 65 104 A1 mentioned at the outset, for example. As a feature of this H-bridge, a diode arrangement 2.9, 2.10 is arranged anti-parallel with respect to two switching members 2,3, 2.4, one of which is arranged in the H-leg leading to the positive pole and one in the oppositely located H-leg leading to the negative pole, in order to assure dependable functioning of the switching members 2.1, 2.2, 2.3, 2.4 during bi-phased operation by free-wheeling, particularly if switching processes at a higher frequency, for example 10 kHz or more, are performed for regulating the pulse energy, for example by a current control. In the transverse branch QZ of the H-bridge 2, an inductive resistor L1 in the form of a coil or of an equivalent switching element lies in series with a coupling relay 3.1 of the coupling circuit 3, as well as in series with a discharge resistor RD or, selectively instead of the latter, with patient electrode connectors PEA, which can be automatically connected by the relay and to which patient electrodes PE are connected during the operation. The discharge resistor RD is, for example, a resistor comparable with the patient impedance of a defined value, for example in the range between 10 and 100 Ohm, for example 25 or 50 Ohm. For assuring a defined state, the energy storage element C can be discharged via the discharge resistor after a time of non-use which can be fixed, or after a defibrillation.

Besides the relay 3.1, the coupling circuit 3 has a further relay 3.2 located between the first one and the patient electrode connectors PEA, by which the patient electrodes PE can be selectively connected with an EKG measuring device EKG or the H-bridge 2 of the high-voltage element. Both relays 3.1, 3.2 can be triggered by respectively assigned relay controls 3.3, 3.4, wherein in the embodiment represented, the relay control 3.3 has control circuit resistors R4, R5, a control circuit transistor T1, as well as a control circuit diode D4 in the arrangement. The further relay control 3.4 has control circuit resistors R6, R7, R8, two control circuit transistors T2, T3, as well as a control circuit diode in the arrangement.

The two relays 3.1, 3.2 can be switched between the indicated switching positions by the relay controls 3.3, 3.4, which can also be designed in a different way.

The coupling circuit 3 is equipped with a relay testing device for the coupling relay 3.1. The relay testing device is used for checking the relay 3.1, wherein the current is cut off from the high-voltage element, in this case the H-bridge 2, by the appropriate triggering of the switching members 2.1, 2.2, 2.3, 2.4. In place of this, a test supply voltage UT, separately provided for the relay test, on an order of magnitude of some, or some 10 Volt, for example between 6 and 30 Volt, is made available by a test supply voltage device. The relay testing device is designed so that, with the discharge resistor RD connected to the relay 3.1, a closed electrical circuit is formed from the positive pole of the test supply voltage UT via a first resistor arrangement RI with several resistors, the discharge resistors RD, the corresponding contacts of the relay 3.1 and a further resistor arrangement RII with several resistors, to ground GND. Parallel with this current path, a measuring branch ME with measuring circuit resistors R1, R2, R3 and measuring circuit diodes D1, D2, D3 and a comparator circuit K is formed between the positive pole of the test supply voltage UT and the first resistor arrangement RI, wherein the measuring circuit resistor R1 is located in a current path leading to an input connector of the comparator circuit K, the measuring circuit resistor R2 is connected to ground between the first measuring circuit resistor R2 and the input connector of the comparator circuit K, and the third measuring circuit resistor R at the output of the comparator circuit K is connected with a preselected potential. The measuring circuit diodes D1, D2 can be stabilizing Zener diodes and are connected to ground upstream of the first measuring circuit resistor R1, while the third measuring circuit diode D3 is connected to ground parallel with the second measuring circuit resistor R2. A preselected or preselectable comparison voltage is connected to the second input connector of the comparator circuit K.

If the discharge resistor RD with the relay 3.1 is connected to the respective current path, a partial current 14 of a total current 11 present from the positive pole of the test supply voltage UT to the branching point ME, flows through the relay 3.1, while a further partial current flows to ground as the measuring current 13 through the measuring branch ME via the second measuring circuit resistor R2 and generates a corresponding voltage drop at the second measuring circuit resistor R2 which, in the connected state of the relay 3.1 results in a comparative voltage corresponding to this state.

If, by an appropriate triggering of the relay 3.1, the discharge resistor RD is not connected, the total current 11 flows as the measuring current 12 through the measuring branch ME and generates a correspondingly greater voltage drop at the second measuring circuit resistor R2, which can be detected by the comparator circuit K. It is thus possible with the measuring branch ME to differentiate between a connected and a non-connected status of the discharge resistor RD, and conclusions can thus be drawn regarding the proper functioning of the relay 3.1, for which purpose the output signal of the comparator circuit K is suitably evaluated in an evaluating circuit.

Other amplification circuits are conceivable in the measuring branch ME wherein, for example, it is possible to differentiate between not completely closed and completely open states of the relay 3.1, provided such an evaluation is desired. Because of the separate test supply voltage UT, the relay testing device is independent of the high-voltage element, so that a dependable statement regarding the ability of the relay to function is possible. The control circuit of the defibrillator is designed so that it connects the high-voltage element with the patient electrodes PE only if the relay 3.1 functions correctly. For example, this function can also be realized in that the further relay 3.2 is triggered so that it interrupts the connection between the patient electrodes PE, or the patient electrode connectors PEA, and the high-voltage element if a faulty state of the coupling relay 3.1 is detected. The evaluation can take place in a suitable logic circuit, for example a programmed micro-controller, or another programmable logic unit (CPLD), and can be used for further control functions.

1. A defibrillator, having an output stage with a high-voltage element (1, 2) and automatically connectable patient electrode connectors (PEA) by a coupling circuit (3) via a relay (3.1) as and a relay testing device, the defibrillator comprising:
a discharge resistor arrangement (RD) to which a switch can be automatically made by the relay (3.1) in lieu of the patient electrode connectors (PEA), and
the relay testing device testing the relay (3.1) while incorporating the status of a connected discharge resistor arrangement (RD).

2. The defibrillator in accordance with claim 1, wherein the relay testing device has a voltage supply for a test supply voltage (UT) by which a current (4) can be run through the relay (3.1) for testing the relay (3.1) with the connected discharge resistor arrangement (RD), and a current from the high-voltage element (1, 2) is blocked.

3. The defibrillator in accordance with claim 2, wherein the relay testing device has a measuring branch (ME) and with one of the discharge resistor arrangement (RD) connected and the discharge resistor arrangement (RD) disconnected, different one of voltages and measuring currents (11, 12) are incorporated in the testing of the relay (3.1).

4. The defibrillator in accordance with claim 3, wherein the measuring branch (ME) has a measurement amplification circuit for forming a measured value regarding a relay status.

5. The defibrillator in accordance with claim 4, wherein the measurement amplification circuit has a comparator for comparing with a reference variable.

6. The defibrillator in accordance with claim 5, wherein the high-voltage element has an H-bridge (2) which is chargeable by an energy storage device (C) with a high voltage for a defibrillation pulse, and has controllable switching members (2.1, 2.2, 2.3, 2.4) in the H-legs, and the relay (3.1) is arranged in series with an inductive resistor (L1) and one of on one side with the discharge resistor (RD) and on an other side with the patient electrodes (PE) connected to the patient electrode connectors (PEA) in the transverse branch (QZ) of the H-bridge (2).

7. The defibrillator in accordance with claim 6, wherein a further relay (3.2) is integrated between the relay (3.1) and the patient electrode connectors (PEA) by which the patient electrodes (PE) are selectively connected with the high-voltage element (1, 2) or an EKG measuring device (EKG).

8. The defibrillator in accordance with claim 1, wherein the relay testing device has a measuring branch (ME) and with one of the discharge resistor arrangement (RD) connected and the discharge resistor arrangement (RD) disconnected, different one of voltages and measuring currents (11, 12) are incorporated in the testing of the relay (3.1).

9. The defibrillator in accordance with claim 8, wherein the measuring branch (ME) has a measurement amplification circuit for forming a measured value regarding a relay status.

10. The defibrillator in accordance with claim 9, wherein the measurement amplification circuit has a comparator for comparing with a reference variable.

11. The defibrillator in accordance with claim 1, wherein the high-voltage element has an H-bridge (2) which is chargeable by an energy storage device (C) with a high voltage for a defibrillation pulse, and has controllable switching members (2.1, 2.2, 2.3, 2.4) in the H-legs, and the relay (3.1) is arranged in series with an inductive resistor (L1) and one of on one side with the discharge resistor (RD) and on an other side with the patient electrodes (PE) connected to the patient electrode connectors (PEA) in the transverse branch (QZ) of the H-bridge (2).

12. The defibrillator in accordance with claim 1, wherein a further relay (3.2) is integrated between the relay (3.1) and the patient electrode connectors (PEA) by which the patient electrodes (PE) are selectively connected with the high-voltage element (1, 2) or an EKG measuring device (EKG).
